(19) United States
(12) Patent Application Publication
(10) Pub. No.: US 2024/0352112 A1
(43) Pub. Date: Oct. 24, 2024

(54) C-TERMINAL ANTIBODY VARIANTS

(71) Applicant: AMGEN INC., Thousand Oaks, CA (US)

(72) Inventors: Zhe Huang, Action, MA (US); Jennitte LeAnn Stevens, Thousand Oaks, CA (US); Greg Flynn, Thousand Oaks, CA (US); Szilan Fodor, Thousand Oaks, CA (US); Mark Daris, Thousand Oaks, CA (US)

(21) Appl. No.: 18/525,130

(22) Filed: Nov. 30, 2023

Related U.S. Application Data
(63) Continuation of application No. 17/934,585, filed on Sep. 23, 2022, now Pat. No. 11,858,983.
Application No. 17/934,585 is a continuation of application No. 16/971,590, filed on Aug. 20, 2020, now Pat. No. 11,466,079.
(60) Provisional application No. 62/812,741, filed on Mar. 1, 2019.
(60) Provisional application No. 62/650,762, filed on Mar. 30, 2018.

(51) Int. Cl. C07K 16/22; A61K 39/00; A61K 47/12; A61K 47/26

(52) U.S. Cl. CPC C07K 16/22; A61K 47/12; A61K 47/26; A61K 2039/505; C07K 2317/24

(57) ABSTRACT

The invention generally relates to anti-sclerostin antibodies having C-terminal modifications, and compositions comprising such antibodies.

### CROSS-REFERENCE TO RELATED APPLICATIONS

The present application claims the benefit of priority to U.S. Provisional Application No. 62/650,762, filed Mar. 30, 2018 and U.S. Provisional Application No. 62/812,741, filed Mar. 1, 2019, the disclosures of which are incorporated herein by reference in their entireties.

### TECHNICAL FIELD OF THE INVENTION

The invention generally relates to anti-sclerostin antibodies having at least one C-terminal modification and compositions comprising such antibodies.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: ASCII (text) file named “52080_SeqListing.txt,” 21,006 bytes, created on Mar. 21, 2019.

### INCORPORATION BY REFERENCE

The following applications are hereby incorporated by reference in their entirety: International Patent Publication No. PCT/US2012/049331, filed Aug. 2, 2012, which claims priority to U.S. Provisional Patent Application No. 61/515,191, filed Aug. 4, 2011; U.S. patent application Ser. No. 11/410,540, filed Apr. 25, 2006, which claims priority to U.S. Provisional Patent Application No. 60/792,645, filed Apr. 17, 2006, U.S. Provisional Patent Application No. 60/782,244, filed Mar. 13, 2006, U.S. Provisional Patent Application No. 60/776,847, filed Feb. 24, 2006, and U.S. Provisional Patent Application No. 60/677,583, filed May 3, 2005; and U.S. patent application Ser. No. 11/411,003 (issued as U.S. Pat. No. 7,592,429), filed Apr. 25, 2006, which claims priority to U.S. Provisional Patent Application No. 60/792,645, filed Apr. 17, 2006, U.S. Provisional Patent Application No. 60/782,244, filed Mar. 13, 2006, U.S. Provisional Patent Application No. 60/776,847, filed Feb. 24, 2006, and U.S. Provisional Patent Application No. 60/677,583, filed May 3, 2005. The following applications also are hereby incorporated by reference: U.S. patent application Ser. No. 12/212,327, filed Sep. 17, 2008, which claims priority to U.S. Provisional Patent Application No. 60/973,024, filed Sep. 17, 2007; and U.S. patent application Ser. No. 12/811,171, filed Jun. 29, 2010, which is a U.S. National Phase Application pursuant to 35 U.S.C. § 371 of International Patent Application No. PCT/US08/86864, filed on Dec. 15, 2008, which claims priority to U.S. Provisional Patent Application No. 61/013,917, filed Dec. 14, 2007.

### BACKGROUND

Loss of bone mineral content can be caused by a wide variety of conditions and may result in significant medical problems. For example, osteoporosis is a debilitating disease in humans and is characterized by marked decreases in skeletal bone mass and mineral density, structural deterioration of bone, including degradation of bone microarchitecture and corresponding increases in bone fragility (i.e., decreases in bone strength), and susceptibility to fracture in afflicted individuals. Although osteoporosis has been regarded as an increase in the risk of fracture due to decreased bone mass, few of the presently available treatments for skeletal disorders can increase the bone density of adults, and most of the presently available treatments work primarily by inhibiting further bone resorption rather than stimulating new bone formation. Estrogen is now being prescribed to retard bone loss. However, some controversy exists over whether patients gain any long-term benefit and whether estrogen has any effect on patients over 75 years old. Moreover, use of estrogen is believed to increase the risk of breast and endometrial cancer. Calcitonin, osteocalcin with vitamin K, or high doses of dietary calcium, with or without vitamin D, have also been suggested for postmenopausal women. High doses of calcium, however, often have undesired gastrointestinal side effects, and serum and urinary calcium levels must be continuously monitored (e.g., Khosla and Riggs, Mayo Clin. Proc. 70:978982, 1995). Other current therapeutic approaches to osteoporosis include bisphosphonates (e.g., Fosamax™, Actonel™, Bonviva™, Zometa™, olpadronate, neridronate, skelid, bonefos), parathyroid hormone, calcilytics, calcimimetics (e.g., cinacalcet), statins, anabolic steroids, lanthanum and strontium salts, and sodium fluoride. Such therapeutics, however, are often associated with undesirable side effects (see Khosla and Riggs, supra).

Sclerostin, the product of the SOST gene, is absent in sclerosteosis, a skeletal disease characterized by bone overgrowth and strong dense bones (Brunkow et al., Am. J. Hum. Genet., 68:577-589, 2001; Balemans et al., Hum. Mol. Genet., 10:537-543, 2001). The amino acid sequence of human sclerostin is reported by Brunkow et al. ibid and is disclosed herein as SEQ ID NO:1. Sclerostin is valuable target for mediating increases in bone density.

### SUMMARY

In one aspect, described herein is an antibody that specifically binds to sclerostin of SEQ ID NO: 1 and comprises a set of six CDRs set forth in SEQ ID NOs: 2-7, wherein the antibody comprises a heavy chain comprising the amino acid sequence Pro-Ala-Arg-Gly (SEQ ID NO: 8) at the C-terminus of the heavy chain. In some embodiments, the antibody comprises a light chain variable region comprising an amino acid sequence set forth in SEQ ID NO: 9 and a heavy chain variable region comprising an amino acid sequence set forth in SEQ ID NO: 10. In some embodiments, the antibody comprises the amino acid sequence Pro-Ala-Arg-Gly-Lys (SEQ ID NO: 11) at the C-terminus of the heavy chain. In some embodiments, the antibody comprises a first heavy chain comprising the amino acid sequence Pro-Ala-Arg-Gly (SEQ ID NO: 8) at the C-terminus of the first heavy chain and a second heavy chain comprising a wild-type heavy chain amino acid sequence (i.e., lacking the C-terminal Pro-Ala-Arg-Gly). In some embodiments, the antibody comprises the light chain amino acid sequence set forth in SEQ ID NO: 12 and the heavy chain amino acid sequence set forth in SEQ ID NO: 13. In some embodiments, the antibody comprises the amino acid sequence Pro-Ala-Arg-Gly-Lys (SEQ ID NO: 11) at the C-terminus of the heavy chain. In some embodiments, the antibody comprises the light chain amino acid sequence set forth in SEQ ID NO: 12 and the heavy chain amino acid sequence set forth in SEQ ID NO: 14.

In some embodiments, the C-terminus of one of the heavy chains of the antibody is amidated (i.e., the antibody is singly amidated). In some embodiments, the C-terminus of both heavy chains of the antibody is amidated (i.e., the antibody is double amidated).

Pharmaceutical compositions comprising a population of the antibodies described herein and a pharmaceutically acceptable carrier are also provided by the disclosure. In some embodiments, the pharmaceutical composition comprises a mixture of antibodies that specifically bind to sclerostin of SEQ ID NO: 1, wherein the mixture of antibodies comprises a population of antibodies comprising a heavy chain having the amino acid sequence Pro-Ala-Arg-Gly (SEQ ID NO: 8) at the C-terminus of the heavy chain and a pharmaceutically acceptable carrier. In some embodiments, about 3-5% of the antibodies in the composition are a population of the antibodies comprising a heavy chain having the amino acid sequence Pro-Ala-Arg-Gly (SEQ ID NO: 8) at the C-terminus of the heavy chain. In some aspects, less than 70% of the population of antibodies are amidated on one or both heavy chains. In some aspects, all or part of the population of antibodies comprise a single heavy chain comprising a C-terminal Pro-Ala-Arg-Gly (SEQ ID NO: 8) sequence, which is optionally amidated. In some aspects, all or part of the population of antibodies comprise a C-terminal Pro-Ala-Arg-Gly (SEQ ID NO: 8) sequence in both heavy chains, and both heavy chains are optionally amidated. Optionally, less than about 35% of the population of antibodies is singly amidated and/or less than about 35% of the population of antibodies are amidated on both heavy chains and/or less than about 35% of the population of antibodies comprise heavy chains that are not amidated. In this regard, in various aspects, about 33% of the population of antibodies are not amidated, about 33% of the population of antibodies comprise one amidated heavy chain, and about 33% of the population of antibodies comprise two amidated heavy chains.

In some embodiments, the composition further comprises a calcium salt, an acetate buffer, a polyol and a surfactant. In some embodiments, the acetate salt comprises calcium acetate, the acetate buffer comprises sodium acetate, the polyol comprises sucrose and the surfactant comprises polysorbate 20. In some embodiments, the composition comprises 55 mM acetate, 13 mm calcium, 6.0% (w/v) sucrose, and 0.006% (w/v) polysorbate 20, pH 5.2.

Also provided by the disclosure is a method of increasing bone mineral density in a subject in need thereof comprising administering the composition described herein to the subject in an amount effective to increase bone mineral density in the subject.

### BRIEF DESCRIPTION OF THE FIGURES

FIG. 1 provides a nucleic acid sequence which encodes a portion of the C-terminus of wild-type romosozumab.

FIG. 2 provides a nucleic acid sequence which encodes a portion of the C-terminus of the romosozumab C-terminal variant (PARG variant).

FIG. 3 is a graph showing the zoomed in UV profile of wild-type romosozumab (dotted line) overlaid with and the romosozumab PARG variant (solid line) that has been digested by Lys-C and analyzed by LC/MS peptide mapping.

FIG. 4 is a graph showing the cation exchange (CEX) profile of carboxypeptidase treated romosozumab PARG variant (dotted line) overlaid with untreated romosozumab PARG variant (solid line).

FIG. 5 is a graph showing the percent recovery from the Scissor model of a subcutaneous injection shown as a function of time. Wild type romosozumab (circles) and PARG c-terminal variant romosozumab diffuse at different rates for the simulated injection site.

FIG. 6 is a graph showing that both wild type romosozumab and PARG C-terminal variant romosozumab bound FcRn similarly and that FcRn binding was not affected by the PARG mutation.

FIG. 7 is a graph showing that the relative binding of PARG C-terminal variant romosozumab to FcγRIIa (131H) was much higher than wild-type romosozumab.

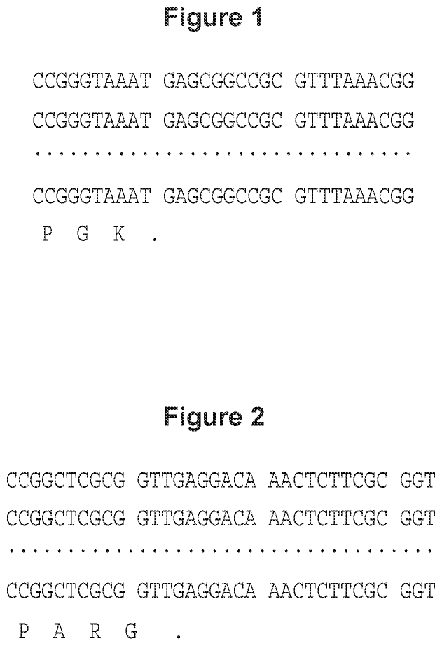

### DETAILED DESCRIPTION

The disclosure provides an antibody that specifically binds to sclerostin, wherein the antibody comprises a heavy chain comprising a sequence of amino acids comprising Pro-Ala-Arg-Gly (SEQ ID NO: 8) at the C-terminus of the heavy chain. In some embodiments, the antibody comprises a first heavy comprising a sequence of amino acids comprising Pro-Ala-Arg-Gly (SEQ ID NO: 8) at the C-terminus of the heavy chain and a second heavy chain comprising a wild-type heavy chain amino acid sequence. In some embodiments, the antibody comprises a sequence of amino acids comprising Pro-Ala-Arg-Gly-Lys (SEQ ID NO: 11) at the C-terminus of the heavy chain. Pharmaceutical compositions comprising the antibody (or mixture of antibodies) and methods of using the antibody are also provided.

An “anti-sclerostin antibody” or an “antibody that binds to sclerostin” is an antibody that binds to sclerostin of SEQ ID NO: 1 or portions thereof. Recombinant human sclerostin/SOST is commercially available from, e.g., R&D Systems (Minneapolis, Minn., USA; 2006 Catalog #1406-ST-025). U.S. Pat. Nos. 6,395,511 and 6,803,453, and U.S. Patent Publication Nos. 2004/0009535 and 2005/0106683 refer to anti-sclerostin antibodies generally. Examples of sclerostin antibodies suitable for use in the context of the invention also are described in U.S. Patent Publication Nos. 2007/0110747 and 2007/0072797, which are hereby incorporated by reference. Additional information regarding materials and methods for generating sclerostin antibodies can be found in U.S. Patent Publication No. 20040158045 (hereby incorporated by reference).

The term “antibody” refers to an intact immunoglobulin molecule (including polyclonal, monoclonal, chimeric, humanized, and/or human versions having full length heavy and/or light chains).

“Specifically binds” as used herein means that the antibody preferentially binds the antigen over other proteins. In some embodiments, “specifically binds” means the antibody has a higher affinity for the antigen than for other proteins. Antibodies that specifically bind an antigen may have a binding affinity for the antigen of less than or equal to 1×10⁻⁷ M, less than or equal to 2×10⁻⁷ M, less than or equal to 3×10⁻⁷ M, less than or equal to 4×10⁻⁷ M, less than or equal to 5×10⁻⁷ M, less than or equal to 6×10⁻⁷ M, less than or equal to 7×10⁻⁷ M, less than or equal to 8×10⁻⁷ M, less than or equal to 9×10⁻⁷ M, less than or equal to 1×10⁻⁸ M, less than or equal to 2×10⁻⁸ M, less than or equal to 3×10⁻⁸ M, less than or equal to 4×10⁻⁸ M, less than or equal to 5×10⁻⁸ M, less than or equal to 6×10⁻⁸ M, less than or equal to 7×10⁻⁸ M, less than or equal to 8×10⁻⁸ M, less than or equal to 9×10⁻⁸ M, less than or equal to 1×10⁻⁹ M, less than or equal to 2×10⁻⁹ M, less than or equal to 3×10⁻⁹ M, less than or equal to 4×10⁻⁷ M, less than or equal to 5×10⁻⁹ M, less than or equal to 6×10⁻⁹ M, less than or equal to 7×10⁻⁷ M, less than or equal to 8×10⁻⁹ M, less than or equal to 9×10⁻⁹ M, less than or equal to 1×10⁻¹⁰ M, less than or equal to 2×10⁻¹⁰ M, less than or equal to 3×10⁻¹⁰ M, less than or equal to 4×10⁻¹⁰ M, less than or equal to 5×10⁻¹⁰ M, less than or equal to 6×10⁻¹⁰ M, less than or equal to 7×10⁻¹⁰ M, less than or equal to 8×10⁻¹⁰ M, less than or equal to 9×10⁻¹⁰ M, less than or equal to 1×10⁻¹¹ M, less than or equal to 2×10⁻¹¹ M, less than or equal to 3×10⁻⁷ M, less than or equal to 4×10⁻¹¹ M, less than or equal to 5×10⁻¹¹ M, less than or equal to 6×10⁻¹¹ M, less than or equal to 7×10⁻¹¹ M, less than or equal to 8×10⁻¹¹ M, less than or equal to 9×10⁻¹¹ M, less than or equal to 1×10⁻¹² M, less than or equal to 2×10⁻¹² M, less than or equal to 3×10⁻¹² M, less than or equal to 4×10⁻¹² M, less than or equal to 5×10⁻¹² M, less than or equal to 6×10⁻¹² M, less than or equal to 7×10⁻¹² M, less than or equal to 8×10⁻¹² M, or less than or equal to 9×10⁻¹² M.

In some or any embodiments, the antibody binds to sclerostin of SEQ ID NO: 1, or a naturally occurring variant thereof, with an affinity (Kd) of less than or equal to 1×10⁻⁷ M, less than or equal to 1×10⁻⁷ M, less than or equal to 1×10⁻⁹ M, less than or equal to 1×10⁻¹⁰ M, less than or equal to 1×10⁻¹¹ M, or less than or equal to 1×10⁻¹² M. Affinity is determined using a variety of techniques, an example of which is an affinity ELISA assay. In various embodiments, affinity is determined by a BIAcore assay. In various embodiments, affinity is determined by a kinetic method. In various embodiments, affinity is determined by an equilibrium/solution method. U.S. Patent Publication No. 2007/0110747 (the disclosure of which is incorporated herein by reference) contains additional description of affinity assays suitable for determining the affinity (Kd) of an antibody for sclerostin.

In some or any embodiments, the antibody (or antibody fragments thereof) binds to a sclerostin polypeptide comprising the amino acid sequence set forth in SEQ ID NO: 1 and binds a region of sclerostin comprising the sequence of SEQ ID NO: 5 (CGPARLLPNAIGRGKWWRPSGPDFRC; corresponding to amino acids 86-111 of SEQ ID NO: 1). This region is also referred to herein as the “loop 2” region of sclerostin. Regions of sclerostin outside of the loop 2 region are defined herein as “non-loop 2 regions.” Alternatively or in addition, the anti-sclerostin antibody binds to a sclerostin polypeptide comprising amino acids 57-146 of SEQ ID NO: 1. Alternatively or in addition, the anti-sclerostin antibody binds to a sclerostin polypeptide comprising amino acids 89-103 of SEQ ID NO: 1 and/or amino acids 137-151 of SEQ ID NO: 1. In some or any embodiments, the sclerostin polypeptide that is a fragment of full length sclerostin retains the tertiary structure of the corresponding polypeptide region of human sclerostin of SEQ ID NO: 1.

In some or any embodiments, the anti-sclerostin antibody described herein preferably modulates sclerostin function in the cell-based assay described in U.S. Patent Publication No. 2007/0110747 and/or the in vivo assay described in U.S. Patent Publication No. 20070110747 and/or bind to one or more of the epitopes described in U.S. Patent Publication No. 2007/0110747 and/or cross-block the binding of one of the antibodies described in U.S. Patent Publication No. 2007/0110747 and/or are cross-blocked from binding sclerostin by one of the antibodies described in U.S. Patent Publication No. 2007/0110747 (incorporated by reference in its entirety and for its description of assays for characterizing an anti-sclerostin antibody).

“CDR” refers to the complementarity determining region within antibody variable sequences. There are three CDRs in each of the variable regions of the heavy chain and the light chain, which are designated CDR1, CDR2 and CDR3, for each of the variable regions. The term “set of six CDRs” as used herein refers to a group of three CDRs that occur in the light chain variable region and heavy chain variable region, which are capable of binding the antigen. The exact boundaries of CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262 (5): 73245 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

CDRs are obtained by, e.g., constructing polynucleotides that encode the CDR of interest. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, “Genetic Manipulation of Monoclonal Antibodies,” in Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)).

In various aspects, the antibody comprises at least one CDR sequence having at least 75% identity (e.g., at least 75%, 80%, 85%, 90%, 95% or 100% identity) to a CDR selected from CDR-H1, CDR-H2, CDR-H3, CDR-L1, CDR-L2, and CDR-L3 wherein CDR-H1 has the sequence given in SEQ ID NO: 2, CDR-H2 has the sequence given in SEQ ID NO: 3, CDR-H3 has the sequence given in SEQ ID NO: 4, CDR-L1 has the sequence given in SEQ ID NO: 5, CDR-L2 has the sequence given in SEQ ID NO: 6 and CDR-L3 has the sequence given in SEQ ID NO: 7. The anti-sclerostin antibody, in various aspects, comprises two of the CDRs or six of the CDRs.

In a preferred embodiment, the anti-sclerostin antibody comprise a set of six CDRs as follows: CDR-H1 of SEQ ID NO: 2, CDR-H2 of SEQ ID NO: 3, CDR-H3 of SEQ ID NO: 4, CDR-L1 of SEQ ID NO: 5, CDR-L2 of SEQ ID NO: 6 and CDR-L3 of SEQ ID NO: 7.

In some or any embodiments, the antibody comprises a light chain variable region comprising an amino acid sequence having at least 75% identity (e.g., at least 75%, 80%, 85%, 90%, 95% or 100% identity) to the amino acid sequence set forth in SEQ ID NO: 9 and a heavy chain variable region comprising an amino acid sequence having at least 75% identity (e.g., at least 75%, 80%, 85%, 90%, 95% or 100% identity) to the amino acid sequence set forth in SEQ ID NO: 10. In various aspects, the difference in the sequence compared to SEQ ID NO: 9 or 10 lies outside the CDR region in the corresponding sequences. In some or any embodiments, the antibody comprises a light chain variable region comprising an amino acid sequence set forth in SEQ ID NO: 9 and a heavy chain variable region comprising an amino acid sequence set forth in SEQ ID NO: 10.

In some or any embodiments the anti-sclerostin antibody comprises all or part of a heavy chain (e.g., two heavy chains) comprising an amino acid sequence having at least 75% identity (e.g., at least 75%, 80%, 85%, 90%, 95% or 100% identity) to the amino acid sequence set forth in SEQ ID NO: 16 and all or part of a light chain (e.g., two light chains) comprising an amino acid sequence having at least 75% identity (e.g., at least 75%, 80%, 85%, 90%, 95% or 100% identity) to the amino acid sequence set forth in SEQ ID NO 12.

The antibody comprises a heavy chain comprising the amino acid sequence Pro-Ala-Arg-Gly (SEQ ID NO: 8) at the C-terminus of the heavy chains. In some embodiments, the C-terminus of both heavy chains of the antibody comprises the amino acid sequence Pro-Ala-Arg-Gly (SEQ ID NO: 8). In some embodiments, the antibody comprises a first heavy chain comprising the amino acid sequence Pro-Ala-Arg-Gly (SEQ ID NO: 8) and a second heavy chain comprising a wild-type amino acid sequence. The antibody, in various aspects, comprises the light chain amino acid sequence set forth in SEQ ID NO: 12 and the heavy chain amino acid sequence set forth in SEQ ID NO: 13.

Alternatively, in some or any embodiments, the antibody comprises a sequence of amino acids comprising Pro-Ala-Arg-Gly-Lys (SEQ ID NO: 11) at the C-terminus of a heavy chain, optionally at the C-terminus of both heavy chains. In some embodiments, the antibody comprises a first heavy chain comprising the amino acid sequence Pro-Ala-Arg-Gly-Lys (SEQ ID NO: 11) and a second heavy chain comprising a wild-type amino acid sequence (i.e., without the C-terminal Pro-Ala-Arg-Gly-Lys (SEQ ID NO: 11)). The antibody, in various aspects, comprises the light chain amino acid sequence set forth in SEQ ID NO: 12 and the heavy chain amino acid sequence set forth in SEQ ID NO: 14.

Examples of other anti-sclerostin antibodies include, but are not limited to, the anti-sclerostin antibodies disclosed in International Patent Publication Nos. WO 2008/092894, WO 2008/115732, WO 2009/056634, WO 2009/047356, WO 2010/100200, WO 2010/100179, WO 2010/115932, and WO 2010/130830 (each of which is incorporated by reference herein in its entirety).

It will be understood by one skilled in the art that some proteins, such as antibodies, may undergo a variety of posttranslational modifications. The type and extent of these modifications often depends on the host cell line used to express the protein as well as the culture conditions. Such modifications may include variations in glycosylation, methionine oxidation, diketopiperizine formation, aspartate isomerization and asparagine deamidation. A frequent modification is the loss of a carboxy-terminal basic residue (such as lysine or arginine) due to the action of carboxypeptidases (as described in Harris, R J. Journal of Chromatography 705:129-134, 1995).

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, pp. 79-86 [1983], entirely incorporated by reference), acetylation of the N-terminal amine, and amidation of any C-terminal carboxyl group.

In some or any embodiments, the C-terminus of the heavy chain of the antibody, comprising the amino acid sequence Pro-Ala-Arg-Gly (SEQ ID NO: 8), is amidated. In some or any embodiments, both heavy chains of the antibody comprise the amino acid sequence Pro-Ala-Arg-Gly (SEQ ID NO: 8) and both heavy chains are amidated. In some embodiments, the glycine is amidated. Amidation can occur, e.g., as described in Prigg, S. T. et al., “New insights into copper monooxygenases and peptide amidation: structure, mechanism and function”, Cell. Mol. Life Sci. 57 (2000) 1236-1259. The enzyme peptidylglycine α-amidating monooxygenase (PAM) can catalyze the amidation of glycine. PAM has two active domains, peptidylglycine α-hydroxylating monooxygenase (PHM) and peptidyl-α-hydroxylglycine α-amidating lyase (PAL). PHM catalyzes the conversion of peptidylglycine (along with ascorbate and oxygen) to peptidyl α-hydroxylglycine (along with semidehydrogenascorbate and water). In turn, PAL catalyzes the conversion of peptidyl α-hydroxylglycine to an amidated peptide (and glyoxylate).

Amidation of an antibody can be controlled by altering certain conditions during the cell culture process. For example, copper (e.g., in ferric ammonium citrate) and/or oxygen levels may be used to influence amidation levels. It is contemplated that increasing copper concentration (e.g., in the media) or oxygen availability (e.g., during culturing) may increase amidation by impacting the activity of an enzyme such as PHM.

### Pharmaceutical Compositions

The disclosure provides a pharmaceutical composition comprising a population of the antibody described herein together with a pharmaceutically effective diluent, carrier, solubilizer, emulsifier, preservative, and/or adjuvant. Pharmaceutical compositions of the invention include, but are not limited to, liquid, frozen, and lyophilized compositions.

The disclosure also provides a pharmaceutical composition comprising a mixture of antibodies that specifically bind to sclerostin of SEQ ID NO: 1 and a pharmaceutically acceptable carrier, wherein about 3-5% of the antibodies in the composition are a population of antibodies described herein (e.g., antibodies comprising set of six CDRs set forth in SEQ ID NOs: 2-7 and having a heavy chain (or two heavy chains) comprising the amino acid sequence Pro-Ala-Arg-Gly (SEQ ID NO: 8) at the C-terminus of the heavy chain(s)). The disclosure also contemplates compositions comprising alternative amounts (e.g., 5-10%, 1-3%, 3-15%, 2-10%, 4-20%, 1-5%) of the population of antibodies described herein (e.g., antibodies comprising set of six CDRs set forth in SEQ ID NOs: 2-7 and having a heavy chain (or two heavy chains) comprising the amino acid sequence Pro-Ala-Arg-Gly (SEQ ID NO: 8) at the C-terminus of the heavy chain(s)).

In some embodiments, less than 70% of the antibodies of the population (e.g., about 69%, about 68%, about 67%, about 66%, about 65%, about 64%, about 63%, about 62%, about 61%, about 60%, about 59%, about 58%, about 57%, about 56%, about 55%, about 54%, about 53%, about 52%, about 51%, about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, bout 13%, about 12%, about 11%, about 10%, about 9%, about 8%, about 7%, about 6%, about 5%, about 4%, about 3%, about 2%, about 1% or less) comprise a heavy chain comprising a C-terminal Pro-Ala-Arg-Gly (SEQ ID NO: 8) sequence, which is optionally amidated. In some embodiments, less than 35% (e.g., about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, bout 13%, about 12%, about 11%, about 10%, about 9%, about 8%, about 7%, about 6%, about 5%, about 4%, about 3%, about 2%, about 1% or less) of the antibodies of the population comprise a C-terminal Pro-Ala-Arg-Gly (SEQ ID NO: 8) sequence on both heavy chains, where both heavy chains are optionally amidated. It is also contemplated that both heavy chains comprise the C-terminal Pro-Ala-Arg-Gly (SEQ ID NO: 8) sequence but only one of the chains is amidated. In some embodiments, less than 35% (e.g., about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, bout 13%, about 12%, about 11%, about 10%, about 9%, about 8%, about 7%, about 6%, about 5%, about 4%, about 3%, about 2%, about 1% or less) of the antibodies in the composition comprise a C-terminal Pro-Ala-Arg-Gly (SEQ ID NO: 8) sequence that is not amidated. In some embodiments, about 33% of antibodies of the population comprise a C-terminal Pro-Ala-Arg-Gly (SEQ ID NO: 8) sequence that is amidated, about 33% of the antibodies of the population comprise C-terminal Pro-Ala-Arg-Gly (SEQ ID NO: 8) sequences on both heavy chains which are both amidated, and about 33% of the antibodies of the population comprise heavy chain(s) with a C-terminal Pro-Ala-Arg-Gly (SEQ ID NO: 8) sequence but which are not amidated.

In some embodiments, the pharmaceutical composition contains formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine, proline, or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. Sec, REMINGTON'S PHARMACEUTICAL SCIENCES, 18″ Edition, (A. R. Genrmo, ed.), 1990, Mack Publishing Company.

Selection of the particular formulation materials described herein may be driven by, for example, the intended route of administration, delivery format and desired dosage. See, for example, REMINGTON'S PHARMACEUTICAL SCIENCES, supra. The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5, and may further include sorbitol or a suitable substitute therefor. In certain embodiments, the composition may be prepared for storage by mixing the selected composition having the desired degree of purity with optional formulation agents (REMINGTON'S PHARMACEUTICAL SCIENCES, supra) in the form of a lyophilized cake or an aqueous solution. Further, in some embodiments, the antibody or fragment may be formulated as a lyophilizate using appropriate excipients such as sucrose.

The pharmaceutical compositions of the invention can be selected for parenteral delivery. Alternatively, the compositions may be selected for inhalation or for delivery through the digestive tract, such as orally. Preparation of such pharmaceutically acceptable compositions is within the skill of the art. The formulation components are present preferably in concentrations that are acceptable to the site of administration. In certain embodiments, buffers are used to maintain the composition at physiological pH or at a slightly lower pH, typically within a pH range of from about 5 to about 8.

When parenteral administration is contemplated, the therapeutic compositions for use in this invention may be provided in the form of a pyrogen-free, parenterally acceptable aqueous solution comprising the desired antibody or fragment in a pharmaceutically acceptable vehicle. A particularly suitable vehicle for parenteral injection is sterile distilled water in which the antibody or fragment is formulated as a sterile, isotonic solution, properly preserved. In certain embodiments, implantable drug delivery devices may be used to introduce the desired antibody or fragment.

In some or any embodiments, the pharmaceutical composition described herein comprises a calcium salt, an acetate buffer, a polyol and a surfactant. Exemplary calcium salts include, but are not limited to, calcium acetate, calcium carbonate and calcium chloride. In some embodiments, the calcium salt is at a concentration of at least 0.5 mM, at least 1 mM, at least 2 mM, at least 3 mM, at least 4 mM, at least 5 mM, at least 6 mM, at least 7 mM, at least 8 mM, at least 9 mM or at least 10 mM. In certain embodiments, the concentration of calcium salt is not greater than 11 mM, no greater than 12 mM, no greater than 13 mM, no greater than 14 mM, no greater than 15 mM, no greater than 16 mM, no greater than 17 mM, no greater than 18 mM, no greater than 19 mM, no greater than 20 mM, no greater than 21 mM, no greater than 22 mM, no greater than 23 mM, no greater than 24 mM, or no greater than 25 mM. Any range featuring a combination of the foregoing endpoints is contemplated, including but not limited to from about 0.5 mM to about 10 mM, about 5 mM to about 10 mM, or about 5 mM to about 15 mM.

In some embodiments, the pharmaceutical composition comprises an acetate buffer (e.g., sodium acetate) having a concentration ranging from about 0.1 mM to about 1000 mM (1 M). In some embodiments, the concentration of the acetate buffer is at least 5 mM, at least 6 mM, at least 7 mM, at least 8 mM, at least 9 mM, at least 10 mM, at least 15 mM, at least 60 mM, at least 70 mM, at least 80 mM, at least 90 mM, at least 100 mM, at least 200 mM, at least 500 mM, at least 700 mM, or at least 900 mM. In some embodiments, the concentration of the acetate buffer is no greater than 10 mM, no greater than 15 mM, no greater than 20 mM, no greater than 25 mM, no greater than 30 mM, no greater than 35 mM, no greater than 40 mM, no greater than 45 mM, no greater than 50 mM, no greater than 55 mM, no greater than 60 mM, no greater than 65 mM, no greater than 70 mM, no greater than 75 mM, no greater than 80 mM, no greater than 85 mM, no greater than 90 mM, no greater than 95 mM or no greater than 100 mM. Any range featuring a combination of the foregoing endpoints is contemplated, including but not limited to from about 5 mM to about 15 mM, or from about 5 mM to about 10 mM or from about 10 mM to about 25 mM. The buffer is preferably added to a concentration that maintains pH around 5-6 or 5-5.5 or 4.5-5.5. When the calcium salt in the formulation is calcium acetate, in some embodiments, the total concentration of acetate is about 10 mM to about 55 mM, or about 20 mM to about 40 mM.

In some aspects, the pharmaceutical composition comprises a total concentration of acetate that is at least about 10 mM, at least about 15 mM, at least about 20 mM, at least about 25 mM, at least about 30 mM, at least about 35 mM, at least about 40 mM, 45 mM, or 50 mM. In some embodiments, the concentration of acetate is no greater than about 30 mM, 35 mM, 40 mM, 45 mM, 50 mM, 55 mM, 60 mM, 65 mM, 70 mM, 75 mM, 80 mM, 85 mM, or 90 mM. Any range featuring a combination of the foregoing endpoints is contemplated, including but not limited to: about 10 mM to about 50 mM, about 20 mM to about 50 mM, about 20 mM to about 40 mM, about 30 mM to about 50 mM, or about 30 mM to about 75 mM. In some embodiments, the calcium salt is calcium acetate and the acetate buffer is sodium acetate. By way of nonlimiting example, a solution containing 10 mM calcium acetate will have 20 mM acetate anion and 10 mM of calcium cation, because of the divalent nature of the calcium cation, while a solution containing 10 mM sodium acetate will have 10 mM sodium cation and 10 mM acetate anion.

In some embodiments, the total concentration of ions (cations and anions) in solution is at least 10 mM, at least about 15 mM, at least about 20 mM, at least about 25 mM, at least about 30 mM, at least about 35 mM, at least about 40 mM, at least about 45 mM, at least about 50 mM, at least about 55 mM, at least about 60 mM, at least about 65 mM, at least about 70 mM, at least about 75 mM, at least about 80 mM, or at least about 85 mM. In some embodiments, the total concentration of ions is no greater than about 30 mM, no greater than about 35 mM, no greater than about 40 mM, no greater than about 45 mM, no greater than about 50 mM, no greater than about 55 mM, no greater than about 60 mM, no greater than about 65 mM, no greater than about 70 mM, no greater than about 75 mM, no greater than about 80 mM, no greater than about 85 mM, no greater than about 90 mM, no greater than about 95 mM, no greater than about 100 mM, no greater than about 110 mM, no greater than about 120 mM, no greater than about 130 mM, no greater than about 140 mM, no greater than about 150 mM, no greater than about 160 mM, no greater than about 170 mM, no greater than about 180 mM, no greater than about 190 mM or no greater than about 200 mM. Any range featuring a combination of the foregoing endpoints is contemplated, including but not limited to: about 30 mM to about 60 mM, or about 30 mM to about 70 mM, or about 30 mM to about 80 mM, or about 40 mM to about 150 mM, or about 50 mM to about 150 mM. By way of nonlimiting example, a solution of 10 mM calcium acetate will have a 30 mM total concentration of ions (10 mM cations and 20 mM anions).

In some or any embodiments, the pharmaceutical composition comprises a polyol. Polyols encompass a class of excipients that includes sugars (e.g. mannitol, sucrose, sorbitol) and other polyhydric alcohols (e.g., glycerol and propylene glycol). Exemplary polyols include, but are not limited to, propylene glycol, glycerin (glycerol), threose, threitol, erythrose, erythritol, ribose, arabinose, arabitol, lyxose, maltitol, sorbitol, sorbose, glucose, mannose, mannitol, levulose, dextrose, maltose, trehalose, fructose, xylitol, inositol, galactose, xylose, fructose, sucrose, 1,2,6-hexanetriol and the like. Higher order sugars include, but are not limited to, dextran, propylene glycol, or polyethylene glycol. Reducing sugars such as fructose, maltose or galactose oxidize more readily than do non-reducing sugars. Additional examples of sugar alcohols are glucitol, maltitol, lactitol or iso-maltulose. Additional exemplary lyoprotectants include glycerin and gelatin, and the sugars mellibiose, melezitose, raffinose, mannotriose, and stachyose. Examples of reducing sugars include glucose, maltose, lactose, maltulose, iso-maltulose and lactulose. Examples of non-reducing sugars include non-reducing glycosides of polyhydroxy compounds selected from sugar alcohols and other straight chain polyalcohols. Monoglycosides include compounds obtained by reduction of disaccharides such as lactose, maltose, lactulose and maltulose.

In some or any embodiments, the pharmaceutical composition comprises a polyol at a concentration ranging from about 0% to about 40% w/v. In some or any embodiments, the compositions comprise a polyol at concentration of at least 0.5, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, or at least 40% w/v. In some or any embodiments, the composition comprises a polyol at a concentration of about 1, 2, 3, 4, 5, 6, 7, 8, 9% to about 10% w/v. In some or any embodiments, the composition comprises a polyol at a concentration of about 2% to about 6% w/v. In some or any embodiments, the composition comprises a polyol at a concentration of about 4% w/v. In some or any embodiments, the composition comprises a polyol at about 6% w/v.

In some or any embodiments, the pharmaceutical composition comprises a surfactant. Exemplary surfactants include, but are not limited to, anionic, cationic, nonionic, zwitterionic, and amphoteric surfactants including surfactants derived from naturally-occurring amino acids. Anionic surfactants include, but are not limited to, sodium lauryl sulfate, dioctyl sodium sulfosuccinate and dioctyl sodium sulfonate, chenodeoxycholic acid, N-lauroylsarcosine sodium salt, lithium dodecyl sulfate, 1-octanesulfonic acid sodium salt, sodium cholate hydrate, sodium deoxycholate, and glycodeoxycholic acid sodium salt. Cationic surfactants include, but are not limited to, benzalkonium chloride or benzethonium chloride, cetylpyridinium chloride monohydrate, and hexadecyltrimethylammonium bromide. Zwitterionic surfactants include, but are not limited to, CHAPS, CHAPSO, SB3-10, and SB3-12. Non-ionic surfactants include, but are not limited to, digitonin, Triton X-100, Triton X-114, TWEEN-20, and TWEEN-80. In another embodiment, surfactants include, but are not limited to, lauromacrogol 400, polyoxyl 40 stearate, polyoxyethylene hydrogenated castor oil 10, 40, 50 and 60, glycerol monostearate, polysorbate 20, polysorbate 40, polysorbate 60, polysorbate 65 and polysorbate 80, soy lecithin and other phospholipids such as DOPC, DMPG, DMPC, and DOPG; sucrose fatty acid ester, methyl cellulose and carboxymethyl cellulose. In some or any embodiments, the surfactant is polysorbate 20.

Surfactants may be included in the compositions either individually or as a mixture in different ratios. In some or any embodiments, the composition comprises a surfactant at a concentration of about 0% to about 5% w/v (e.g., about 0.001, about 0.002, about 0.005, about 0.007, about 0.01, about 0.05, about 0.1, about 0.2, about 0.3, about 0.4, about 0.5, about 0.6, about 0.7, about 0.8, about 0.9, about 1.0, about 1.5, about 2.0, about 2.5, about 3.0, about 3.5, about 4.0, or about 4.5% w/v). In some or any embodiments, the composition comprises a surfactant at a concentration of about 0.001% to about 0.5% w/v. In some or any embodiments, the composition comprises a surfactant at a a concentration of about 0.004, about 0.005, about 0.007, about 0.01, about 0.05, or about 0.1% w/v to about 0.2% w/v. In some or any embodiments, the composition comprises a surfactant at a concentration of about 0.01% to about 0.1% w/v.

In some or any embodiments, the pharmaceutical composition comprises 55 mM acetate, 13 mm calcium, 6.0% (w/v) sucrose, 0.006% (w/v) polysorbate 20, pH 5.2.

Additional pharmaceutical compositions will be evident to those skilled in the art, including formulations involving antigen binding proteins in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, International Patent Application No. PCT/US93/00829, which is incorporated by reference and describes controlled release of porous polymeric microparticles for delivery of pharmaceutical compositions. Sustained-release preparations may include semipermeable polymer matrices in the form of shaped articles, e.g., films, or microcapsules. Sustained release matrices may include polyesters, hydrogels, polylactides (as disclosed in U.S. Pat. No. 3,773,919 and European Patent Application Publication No. EP058481, each of which is incorporated by reference), copolymers of L-glutamic acid and gamma ethyl-L-glutamate (Sidman et al., 1983, Biopolymers 2:547-556), poly(2-hydroxyethyl-methacrylate) (Langer et al., 1981, J. Biomed. Mater. Res. 15:167-277 and Langer, 1982, Chem. Tech. 12:98-105), ethylene vinyl acetate (Langer et al., 1981, supra) or poly-D(−)-3-hydroxybutyric acid (European Patent Application Publication No. EP133988). Sustained release compositions may also include liposomes that can be prepared by any of several methods known in the art. Sec, e.g., Eppstein et al., 1985, Proc. Natl. Acad. Sci. U.S.A. 82:3688-3692; European Patent Application Publication Nos. EP036676; EP088046 and EP143949, incorporated by reference.

Pharmaceutical compositions used for in vivo administration are typically provided as sterile preparations. Sterilization can be accomplished by filtration through sterile filtration membranes. When the composition is lyophilized, sterilization using this method may be conducted either prior to or following lyophilization and reconstitution. Compositions for parenteral administration can be stored in lyophilized form or in a solution. Parenteral compositions generally are placed into a container having a sterile access port, for example, an intravenous solution bag or vial having a stopper pierceable by a hypodermic injection needle.

Free amino acids can be used in antibody or fragment formulations in accordance with various embodiments of the invention as bulking agents, stabilizers, and antioxidants, as well as other standard uses. Lysine, proline, serine, and alanine can be used for stabilizing proteins in a formulation. Glycine is useful in lyophilization to ensure correct cake structure and properties. Arginine may be useful to inhibit protein aggregation, in both liquid and lyophilized formulations. Methionine is useful as an antioxidant.

Embodiments of antibody formulations may further comprise one or more antioxidants. To some extent deleterious oxidation of proteins can be prevented in pharmaceutical formulations by maintaining proper levels of ambient oxygen and temperature and by avoiding exposure to light. Antioxidant excipients can be used as well to prevent oxidative degradation of proteins. Among useful antioxidants in this regard are reducing agents, oxygen/free-radical scavengers, and chelating agents. Antioxidants for use in therapeutic protein formulations in accordance with the invention preferably are water-soluble and maintain their activity throughout the shelf life of a product. EDTA is a preferred antioxidant in accordance with the invention in this regard.

Formulations in accordance with the invention may include metal ions that are protein co-factors and that are necessary to form protein coordination complexes, such as zinc necessary to form certain insulin suspensions. Metal ions also can inhibit some processes that degrade proteins. However, metal ions also catalyze physical and chemical processes that degrade proteins.

Magnesium ions (10-120 mM) can be used to inhibit isomerization of aspartic acid to isoaspartic acid. Ca⁺² ions (up to 100 mM) can increase the stability of human deoxyribonuclease. Mg⁺², Mn⁺², and Zn⁺², however, can destabilize rhDNase. Similarly, Ca⁺² and Sr⁺² can stabilize Factor VIII, it can be destabilized by Mg⁺², Mn⁺² and Zn⁺², Cu⁺² and Fe⁺², and its aggregation can be increased by Al⁺³ ions.

Embodiments of the antibody formulations can further comprise one or more preservatives.

Once the pharmaceutical composition has been formulated, it may be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration. The invention also provides kits for producing a single-dose administration unit. The kits of the invention may each contain both a first container having a dried protein and a second container having an aqueous formulation. In certain embodiments of this invention, kits containing single and multi-chambered pre-filled syringes (e.g., liquid syringes and lyosyringes) are provided.

The therapeutically effective amount of an antibody-containing pharmaceutical composition to be employed will depend, for example, upon the therapeutic context and objectives. One skilled in the art will appreciate that the appropriate dosage levels for treatment will vary depending, in part, upon the molecule delivered, the indication(s) for which the antibody is being used, the route of administration, and the size (body weight, body surface or organ size) and/or condition (the age and general health) of the patient.

### Stability

The terms “stability” and “stable” as used herein in the context of a composition comprising an antibody (or antigen binding fragment thereof) refer to the resistance of the antibody (or antigen binding fragment thereof) in the composition to aggregation, degradation or fragmentation under given manufacture, preparation, transportation and/or storage conditions. Antibody formulations comprising a high degree of stability demonstrate enhanced reliability and safety and, as such, are advantageous for clinical use.

Antibody stability in a composition is optionally assessed by examining a desired parameter of the antibody in the composition (e.g., aggregation, degradation of heavy and/or light chains, chemical modification, etc.) over time. In this regard, a parameter is typically examined at an initial time point (T0) and an assessment time point (T1), optionally while exposing the antibody to any of a number of environmental conditions, and compared. An initial time point can be, for instance, the time that the antibody is first formulated in a composition or first examined for quality (i.e., examined to determine whether the antibody composition meets regulatory or manufacturing specifications with respect to aggregation or degradation). An initial time point also can be the time at which the antibody is reformulated in a composition (e.g., reformulated at a higher or lower concentration compared to an initial preparation). An assessment time point is, in various embodiments, about 1 week (or about 2 weeks, or about 3 weeks, or about 4 weeks, or about 5 weeks, or about 6 weeks, or about 7 weeks, or about 8 weeks, or about 10 weeks, or about 3 months, or about 6 months or about 1 year) after the initial time point. The desired parameter (e.g., aggregation or degradation) of the antibody or fragment thereof in the composition can be assessed under a variety of storage conditions, such as temperatures of −30° C., 4° C., 20° C. or 40° C., shaking, pH, storage in different container materials (e.g., glass vials, pre-filled syringes, etc.), and the like.

Exemplary methods for determining the degree of aggregation, and/or types and/or sizes of aggregates present in a composition comprising the antibody include, but are not limited to, size exclusion chromatography (SEC), high performance size exclusion chromatography (HPSEC), static light scattering (SLS), Fourier Transform Infrared Spectroscopy (FTIR), circular dichroism (CD), urea-induced protein unfolding techniques, intrinsic tryptophan fluorescence, differential scanning calorimetry, and 1-anilino-8-naphthalenesulfonic acid (ANS) protein binding techniques. Size exclusion chromatography (SEC) may be performed to separate molecules on the basis of their size, by passing the molecules over a column packed with the appropriate resin, the larger molecules (e.g. aggregates) will elute before smaller molecules (e.g. monomers). The molecules are generally detected by UV absorbance at 280 nm and may be collected for further characterization. High pressure liquid chromatographic columns are often utilized for SEC analysis (HP-SEC). Alternatively, analytical ultracentrifugation (AUC) may be utilized. AUC is an orthogonal technique which determines the sedimentation coefficients of macromolecules in a liquid sample. Like SEC. AUC is capable of separating and detecting antibody fragments/aggregates from monomers and is further able to provide information on molecular mass. Antibody aggregation in a composition may also be characterized by particle counter analysis using a coulter counter or by turbidity measurements using a turbidimeter. Turbidity is a measure of the amount by which the particles in a solution scatter light and, thus, may be used as a general indicator of protein aggregation. In addition, non-reducing polyacrylamide gel electrophoresis (PAGE) or capillary gel electrophoresis (CGE) may be used to characterize the aggregation and/or fragmentation state of antibodies or antibody fragments in a composition.

Exemplary methods for determining antibody degradation include, but are not limited to, size-exclusion chromatography (SEC), sodium dodecyl sulfate-polyacrylamide gel electrophoresis (SDS-PAGE) and capillary electrophoresis with SDS (CE-SDS) and reversed phase HPLC with in-line MS detection.

In various embodiments, less than 5% of the antibody described herein in the composition is in aggregate form under conditions of interest. For instance, less than 4%, or less than 3%, or less than 2%, or less than 1% of the antibody in the composition is in aggregate form after storage at −30° C., 4° C., 20° C. or 40° C. for a period of about 1 week (or about 2 weeks, or about 3 weeks, or about 4 weeks, or about 5 weeks, or about 6 weeks, or about 7 weeks, or about 8 weeks, or about 10 weeks, or about 3 months, or about 6 months or about 1 year). In some embodiments, less than 5% (or less than 4% or less than 3% or less than 2% or less than 1% or less) of the antibody described herein in the composition is in aggregate form after storage for two weeks at about 4° C.

For example at least 85% (or at least 90%, or at least 91%, or at least 92%, or at least 93%, or at least 94%, or at least 95%, or at least 96%, or at least 97%, or at least 98%, or at least 99%) of antibody in a composition optionally is present in non-aggregate (i.e., monomeric) form after storage at −30° C., 4° C., 20° C. or 40° C. for a period of about 1 week (or about 2 weeks, or about 3 weeks, or about 4 weeks, or about 5 weeks, or about 6 weeks, or about 7 weeks, or about 8 weeks, or about 10 weeks, or about 3 months, or about 6 months or about 1 year). In some embodiments, at least 85% (or at least 90%, or at least 91%, or at least 92%, or at least 93%, or at least 94%, or at least 95%, or at least 96%, or at least 97%, or at least 98%, or at least 99% or more) of the antibody is present in the composition in non-aggregate form after two weeks of storage at about 4° C. In some embodiments, at least 99% of the antibody is present in the composition in non-aggregate form after storage for two weeks at about 4° C. for two weeks and/or at least 95% of antibody present in the composition is in non-aggregate form after storage for two weeks at 40° C.

In various embodiments, less than 5% of the antibody described herein in the composition is degraded. For instance, less than 4%, or less than 3%, or less than 2%, or less than 1% or less of the antibody in the composition is degraded under conditions of interest. For example, optionally at least 85% (or at least 90%, or at least 91%, or at least 92%, or at least 93%, or at least 94%, or at least 95%, or at least 96%, or at least 97%, or at least 98%, or at least 99%) of the antibody is intact (i.e., not degraded) in a composition stored at about-30° C., about 4° C., about 20° C. or about 40° C. for a period of about 1 week (or about 2 weeks, or about 3 weeks, or about 4 weeks, or about 5 weeks, or about 6 weeks, or about 7 weeks, or about 8 weeks, or about 10 weeks, or about 3 months, or about 6 months or about 1 year). In some aspects, at least 85% (or at least 90%, or at least 91%, or at least 92%, or at least 93%, or at least 94%, or at least 95%, or at least 96%, or at least 97%, or at least 98%, or at least 99% or more) of the antibody is intact (i.e., non-degraded) after storage in a composition at about 4° C. for a period of two weeks. In some embodiments, at least 99% of the antibody remains intact when stored in a composition at about 4° C. for two weeks and/or at least 95% remains intact when stored in a composition at about 40° C. for two weeks.

Functional or activity stability of the antibody in a composition also is contemplated herein. Assays for detecting and/or quantifying, e.g., antibody binding to a target or sclerostin neutralization are known in the art. Optionally, the antibody demonstrates about 50-100% activity under conditions of interest compared to the activity of the antibody at the initial time point. For example, the antibody retains a level of activity of between about 60-90% or 70-80% compared to the activity the initial time point. Accordingly, functional stability of the antibody includes retention of activity of at least about 50%, 55%. 60%, 65%, 70%, 75%, 80%, 85%, 90%, 95% or 100% and can include activity measurements greater than 100% such as 105%, 110%, 115%, 120%, 125% or 150% or more compared to the activity at the initial time point.

### Viscosity

In some embodiments, the viscosity of a composition comprising one or more of the antibodies described herein is determined. The term “viscosity” as used herein refers to “absolute viscosity.” Absolute viscosity, sometimes called dynamic or simple viscosity, is the product of kinematic viscosity and fluid density (Absolute Viscosity=Kinematic Viscosity×Density). The dimension of kinematic viscosity is L²/T where L is a length and T is a time. Commonly, kinematic viscosity is expressed in centistokes (cSt). The SI unit of kinematic viscosity is mm²/s, which is 1 cSt. Absolute viscosity is expressed in units of centipoise (cP). The SI unit of absolute viscosity is the millipascal-second (mPa-s), where 1 cP=1 mPa-s.

The viscosity of a composition can be measured hours (e.g., 1-23 hours), days (e.g., 1-10 days), weeks (e.g., 1-5 weeks), months (e.g., 1-12 months), or years (e.g., 1-2 years, 1-3 years) after the addition of the antibody to the composition. Viscosity measurements may be made at a storage or administration temperature, e.g. 2-8° C. or 25° C. (room temperature). In some embodiments, absolute viscosity of the liquid or reconstituted liquid composition at the storage and/or administration temperature is 15 cP or less, or 14, 13, 12, 11, 10, 9, 8, 7, 6, 5, or 4 cP or less. In some embodiments, absolute viscosity of the liquid or reconstituted liquid composition is 6 cP or less.

In some embodiments, the viscosity of the antibody composition is measured prior to and after the addition of antibody. Methods of measuring viscosity are well known in the art and include, for example, using a capillary viscometer, or a cone-plate rheometer. Any method may be used provided the same method is used to compare the test and reference formulations.

### Therapeutic Methods

The antibody and pharmaceutical compositions described herein are useful for treating or preventing bone-related disorders, such as bone-related disorders associated with abnormal osteoblast or osteoclast activity. In some embodiments, the antibody is administered to a subject suffering from a bone related disorder selected from the group consisting of achondroplasia, cleidocranial dysostosis, enchondromatosis, fibrous dysplasia, Gaucher's Disease, hypophosphatemic rickets, Marfan's syndrome, multiple hereditary exotoses, neurofibromatosis, osteogenesis imperfecta, osteopetrosis, osteopoikilosis, sclerotic lesions, pseudoarthrosis, pyogenic osteomyelitis, periodontal disease, anti-epileptic drug induced bone loss, primary and secondary hyperparathyroidism, familial hyperparathyroidism syndromes, weightlessness induced bone loss, osteoporosis in men, postmenopausal bone loss, osteoarthritis, renal osteodystrophy, infiltrative disorders of bone, oral bone loss, osteonecrosis of the jaw, juvenile Paget's disease, melorheostosis, metabolic bone diseases, mastocytosis, sickle cell anemia/disease, organ transplant related bone loss, kidney transplant related bone loss, systemic lupus erythematosus, ankylosing spondylitis, epilepsy, juvenile arthritides, thalassemia, mucopolysaccharidoses, Fabry Disease, Turner Syndrome, Down Syndrome, Klinefelter Syndrome, leprosy, Perthe's Disease, adolescent idiopathic scoliosis, infantile onset multi-system inflammatory disease, Winchester Syndrome, Menkes Disease, Wilson's Disease, ischemic bone disease (such as Legg-Calve-Perthes disease and regional migratory osteoporosis), anemic states, conditions caused by steroids, glucocorticoid-induced bone loss, heparin-induced bone loss, bone marrow disorders, scurvy, malnutrition, calcium deficiency, osteoporosis, osteopenia, alcoholism, chronic liver disease, postmenopausal state, chronic inflammatory conditions, rheumatoid arthritis, inflammatory bowel disease, ulcerative colitis, inflammatory colitis, Crohn's disease, oligomenorrhea, amenorrhea, pregnancy-related bone loss, diabetes mellitus, hyperthyroidism, thyroid disorders, parathyroid disorders, Cushing's disease, acromegaly, hypogonadism, immobilization or disuse, reflex sympathetic dystrophy syndrome, regional osteoporosis, osteomalacia, bone loss associated with joint replacement, HIV associated bone loss, bone loss associated with loss of growth hormone, bone loss associated with cystic fibrosis, chemotherapy-associated bone loss, tumor-induced bone loss, cancer-related bone loss, hormone ablative bone loss, multiple myeloma, drug-induced bone loss, anorexia nervosa, disease-associated facial bone loss, disease-associated cranial bone loss, disease-associated bone loss of the jaw, disease-associated bone loss of the skull, bone loss associated with aging, facial bone loss associated with aging, cranial bone loss associated with aging, jaw bone loss associated with aging, skull bone loss associated with aging, and bone loss associated with space travel.

In some embodiments, the antibodies described herein are useful for improving outcomes in orthopedic procedures, dental procedures, implant surgery, joint replacement, bone grafting, bone cosmetic surgery and bone repair such as fracture healing, nonunion healing, delayed union healing and facial reconstruction. A composition comprising one or more antibodies may be administered before, during and/or after the procedure, replacement, graft, surgery or repair.

In some embodiments, the antibodies described herein are useful for the treatment of any fracture comprising a gap between two segments of bone (e.g., a gap of at least about 1 mm between two segments of bone). In some or any embodiments, the gap is at least about 2 mm, at least about 3 mm, at least about 4 mm, at least about 5 mm, at least about 6 mm, at least about 7 mm, at least about 8 mm, at least about 9 mm, or at least about 1 cm or more. In some or any embodiments, the gap is about 5 mm to 1 cm, or up to 1 cm. The terms “bone gap defect” and “segmental skeletal defect” are used synonymously herein and refer to a gap between two segments of bone (e.g., a gap of at least 1 mm).

Exemplary bone gap defects include, but are not limited to, a comminuted fracture, a non-union fracture, a segmental skeletal defect, surgically created bone defects, surgically treated bone defects, and bone defects created from traumatic injury to the bone or disease (including, but not limited to, arthritis, tumor removal (resection) or infection removal). In some or any embodiments, the bone gap defect is produced by removal of infected sections of bone or the removal of cancer from the bone due to bone cancers including, but not limited to, osteosarcoma, Ewing's sarcoma, chondrosarcoma, malignant fibrous histiocytoma, fibrosarcoma, and chordoma. In some or any embodiments, the bone gap defect is a developmental deformity, e.g., due to a genetic defect.

In some or any embodiments, the bone gap defect is produced by removal of sections of bone containing a benign tumor. Exemplary benign bone tumors include, but are not limited to, osteoma, osteoid osteoma, osteoblastoma, osteochondroma, enchondroma, chonrdomyxoid fibroma, aneurysmal bone cyst, unicameral bone cyst, fibrous dysplasia of bone and giant cell tumor of the bone.

Administration of the antibody enhances or accelerates bone gap defect healing, thereby “treating” the bone gap defect. “Enhancing” bone healing means mediating a level of bone healing beyond (i.e., greater than) the level of bone healing experienced in subjects (e.g., mammals, such as humans) not administered the sclerostin inhibitor (i.e., control subjects). Bone healing is evidenced by, for example, bridging status, improved bone volume, improved bone mineral content and density within the fracture gap (i.e., formation of bridging bone), mature bone callus, improved bone strength (optionally accompanied by a medically-acceptable level of bone stiffness), or improved patient use of the affected area. By “improved” is meant an increase or decrease (as desired) in the measured parameter. The increase can be a return, in whole or in part, of the measured parameter to baseline level (e.g., the level prior to the bone gap defect), to values provided in normative databases used in the art, or to the contralateral functional level (e.g., return, in whole or in part, to the functional capabilities of, for example, the contralateral limb). In some cases, the increase can be an improvement beyond baseline level. If desired, the measured parameters in patients administered one or more doses of the antibody can be compared to the same parameters in fracture patients (optionally age and gender matched) not administered the antibody to further analyze the efficacy of the methods described herein.

Formation of bridging bone, bone mineral content and bone density, and/or mature boney callus at the site of bone defect may be measured using radiography (e.g., radiographic absorptometry), single- and/or dual-energy X-ray absorptometry, quantitative computed tomography (QCT), ultrasonography, radiography (e.g., radiographic absorptometry), and magnetic resonance imaging. In some embodiments, the antibody may be administered at a dose and for a time period effective to increase bridging bone formation, formation of bony callus, or bone density (or volume) at the defect site by at least about 5% (about 6%, about 7%, about 8%, or about 9%). In some embodiments, bridging bone formation, formation of bony callus, or bone density at the defect site is increased by at least about 10% (e.g., at least about 10%, at least about 12%, at least about 15%, at least about 18%, at least about 20%, or at least about 22%). In other embodiments, bridging bone formation, formation of bony callus, or bone density at the defect site is increased by the sclerostin inhibitor at least about 25% (e.g., at least about 26% or at least about 28%). In yet other embodiments, bridging bone formation, formation of bony callus, or bone density at the defect site is increased at least about 30% (e.g., at least about 32%, at least about 35%, at least about 38%, or at least about 40%) or at least about 50% (e.g., at least about 60%, at least about 70%, at least about 80%, at least about 90%, or about 100%). The increase or re-establishment of bridging bone formation can be determined at 1 week, 2 weeks, 3 weeks, or 4 weeks following the initial administration of antibody. Alternatively, the bone density level can be determined after the treatment period ends (e.g., 1 week, 2 weeks, 3 weeks, or 4 weeks after the treatment period ends). In one aspect, the method reduces the amount of time required to establish a desired level of bone formation, bone volume, bony callus, or bone density (e.g., any percent increase in bone formation, bone mineral density, bony callus, or bone volume described herein) compared to age and gender-matched patients that do not receive the antibody, thereby reducing recovery time for a subject. For example, in one embodiment, the antibody reduces the amount of time required to increase bone density or volume at the defect site at least about 10% (e.g., at least about 20%, at least about 25%, at least about 30%, at least about 35%, at least about 40%, at least about 45%, or at least about 50%).

The antibody need not cure the subject of the disorder or completely protect against the onset of a bone-related disorder to achieve a beneficial biological response. The antibody may be used prophylactically, meaning to protect, in whole or in part, against a bone-related disorder or symptom thereof. The antibody also may be used therapeutically to ameliorate, in whole or in part, a bone-related disorder or symptom thereof, or to protect, in whole or in part, against further progression of a bone-related disorder or symptom thereof. Indeed, the materials and methods of the invention are particularly useful for increasing bone mineral density, and optionally maintaining the increased bone mineral density over a period of time.

In some embodiments, one or more administrations of an antibody described herein are carried out over a therapeutic period of, for example, about 1 week to about 18 months (e.g., about 1 month to about 12 months, about 1 month to about 9 months or about 1 month to about 6 months or about 1 month to about 3 months). In some embodiments, a subject is administered one or more doses of a antibody described herein over a therapeutic period of, for example about 1 month to about 12 months (52 weeks) (e.g., about 2 months, about 3 months, about 4 months, about 5 months, about 6 months, about 7 months, about 8 months, about 9 months, about 10 months, or about 11 months).

In addition, it may be advantageous to administer multiple doses of the antibody or space out the administration of doses, depending on the therapeutic regimen selected for a particular subject. In some embodiments, the antibody or fragment thereof is administered periodically over a time period of one year (12 months, 52 weeks) or less (e.g., 9 months or less, 6 months or less, or 3 months or less). In this regard, the antibody or fragment thereof is administered to the human once every about 3 days, or about 7 days, or 2 weeks, or 3 weeks, or 4 weeks, or 5 weeks, or 6 weeks, or 7 weeks, or 8 weeks, or 9 weeks, or 10 weeks, or 11 weeks, or 12 weeks, or 13 weeks, or 14 weeks, or 15 weeks, or 16 weeks, or 17 weeks, or 18 weeks, or 19 weeks, or 20 weeks, or 21 weeks, or 22 weeks, or 23 weeks, or 6 months, or 12 months.

In some embodiments, one or more doses of the antibody are administered in an amount and for a time effective to increase bone mineral density or treat a bone disorder associated with decreased bone mineral density. In various embodiments, one or more doses comprising from about 50 milligrams to about 1,000 milligrams of the antibody are administered per week to a subject (e.g., a human subject). For example, a dose of antibody can comprise at least about 5 mg, 15 mg, 25 mg. 50 mg, about 60 mg, about 70 mg, about 80 mg, about 90 mg, about 100 mg, about 120 mg, about 150 mg, about 200 mg, about 210 mg, about 240 mg, about 250 mg, about 280 mg, about 300 mg, about 350 mg, about 400 mg, about 420 mg, about 450 mg, about 500 mg, about 550 mg, about 600 mg, about 650 mg, about 700 mg, about 750 mg, about 800 mg, about 850 mg, about 900 mg, about 950 mg or up to about 1,000 mg of antibody. Ranges between any and all of these endpoints are also contemplated, e.g. about 50 mg to about 80 mg, about 70 mg to about 140 mg, about 70 mg to about 270 mg, about 75 mg to about 100 mg, about 100 mg to about 150 mg, about 140 mg to about 210 mg, or about 150 mg to about 200 mg, or about 180 mg to about 270 mg, or about 280 to about 410 mg. The dose is administered at any interval, such as multiple times a week (e.g., twice or three times per week), once a week, once every two weeks, once every three weeks, or once every four weeks. In some or any embodiments, a dose of antibody ranging from about 120 mg to about 210 mg is administered twice a week. In some or any embodiments, a dose of about 140 mg of the antibody is administered twice a week. In various aspects, a dose of about 210 mg of antibody is administered once a month.

In some embodiments, the one or more doses of antibody can comprise between about 0.1 to about 50 milligrams (e.g., between about 5 and about 50 milligrams), or about 1 to about 100 milligrams, of antibody per kilogram of body weight (mg/kg). For example, the dose of antibody may comprise at least about 0.1 mg/kg. 0.5 mg/kg, 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 20 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, or about 49 mg/kg, or about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or up to about 100 mg/kg. Ranges between any and all of these endpoints are also contemplated, e.g., about 1 mg/kg to about 3 mg/kg, about 1 mg/kg to about 5 mg/kg, about 1 mg/kg to about 8 mg/kb, about 3 mg/kg to about 8 mg·kg, about 1 mg/kg to about 10 mg/kg, about 1 mg/kg to about 20 mg/kg, about 1 mg/kg to about 40 mg/kg, about 5 mg/kg to about 30 mg/kg, or about 5 mg/kg to about 20 mg/kg.

### Monitoring Therapy

Antibody-mediated increases in bone mineral content or bone density may be measured using single- and dual-energy X-ray absorptometry, ultrasound, computed tomography, radiography, and magnetic resonance imaging. The amount of bone mass may also be calculated from body weights or by using other methods (see Guinness-Hey, Metab. Bone Dis. Relat. Res., 5:177-181 (1984)). Animal models are used in the art for testing the effect of the pharmaceutical compositions and methods on, for example, parameters of bone loss, bone resorption, bone formation, bone strength, or bone mineralization that mimic conditions of human disease such as osteoporosis and osteopenia. Examples of such models include the ovariectomized rat model (Kalu, Bone and Mineral, 15:175-192 (1991); Frost and Jee. Bone and Mineral, 18:227-236 (1992); and Jee and Yao, J. Musculoskel. Neuron. Interact., 1:193-207 (2001)). The methods for measuring antibody activity described herein also may be used to determine the efficacy of other sclerostin inhibitors.

In humans, bone mineral density can be determined clinically using dual x-ray absorptiometry (DXA) of, for example, the hip and spine. Other techniques include quantitative computed tomography (QCT), ultrasonography, single-energy x-ray absorptiometry (SXA), and radiographic absorptiometry. Common central skeletal sites for measurement include the spine and hip; peripheral sites include the forearm, finger, wrist and heel. Except for ultrasonography, the American Medical Association notes that BMD techniques typically involve the use of x-rays and are based on the principle that attenuation of the radiation depends on thickness and composition of the tissues in the radiation path. All techniques involve the comparison of results to a normative database.

Alternatively, a physiological response to one or more anti-sclerostin antibodies can be gauged by monitoring bone marker levels. Bone markers are products created during the bone remodeling process and are released by bone, osteoblasts, and/or osteoclasts. Fluctuations in bone resorption and/or bone formation “marker” levels imply changes in bone remodeling/modeling. The International Osteoporosis Foundation (IOF) recommends using bone markers to monitor bone density therapies (see, e.g., Delmas et al., Osteoporos Int., Suppl. 6: S2-17 (2000), incorporated herein by reference). Markers indicative of bone resorption (or osteoclast activity) include, for example, C-telopeptide (e.g., C-terminal telopeptide of type 1 collagen (CTX) or serum cross-linked C-telopeptide), N-telopeptide (N-terminal telopeptide of type 1 collagen (NTX)), deoxypyridinoline (DPD), pyridinoline, urinary hydroxyproline, galactosyl hydroxylysine, and tartrate-resistant acid phosphatase (e.g., serum tartrate-resistant acid phosphatase isoform 5b). Bone formation/mineralization markers include, but are not limited to, bone-specific alkaline phosphatase (BSAP), peptides released from N- and C-terminal extension of type I procollagen (PINP, PICP), and osteocalcin (OstCa). Several kits are commercially-available to detect and quantify markers in clinical samples, such as urine and blood.

### Combination Therapy

Treatment of a pathology by combining two or more agents that target the same pathogen or biochemical pathway or biological process sometimes results in greater efficacy and diminished side effects relative to the use of a therapeutically relevant dose of each agent alone. In some cases, the efficacy of the drug combination is additive (the efficacy of the combination is approximately equal to the sum of the effects of each drug alone), but in other cases the effect is synergistic (the efficacy of the combination is greater than the sum of the effects of each drug given alone). As used herein, the term “combination therapy” means that two or more agents are delivered in a simultaneous manner, e.g., concurrently, or wherein one of the agents is administered first, followed by the second agent, e.g., sequentially.

In some embodiments, the antibody is administered along with a standard of care therapeutic for the treatment of decreased bone mineral density (i.e., the antibody and standard of care therapeutic are part of the same treatment plan). As used herein, the term “standard of care” refers to a treatment that is generally accepted by clinicians for a certain type of patient diagnosed with a type of illness. In some embodiments, the antibody is administered along with a second bone-enhancing agent useful for the treatment of decreased bone mineral density or bone defect. In some embodiments, the bone-enhancing agent is selected from the group consisting of an anti-resorptive agent, a bone-forming agent (i.e., anabolic), an estrogen receptor modulator (including, but not limited to, raloxifene, bazedoxifene and lasofoxifene) and a drug that has an inhibitory effect on osteoclasts. In some embodiments, the second bone-enhancing agent is selected from the group consisting of a bisphosphonate (including, but not limited to, alendronate sodium (FOSAMAX®), risedronate, ibandronate sodium (BONIVA®) and zoledronic acid (RECLAST®)); an estrogen or estrogen analogue; an anti-RANK ligand (RANKL) inhibitor, such as an anti-RANKL antibody (e.g., denosumab, PROLIA®); vitamin D, or a vitamin D derivative or mimic thereof; a calcium source, a cathepsin-K (cat-K) inhibitor (e.g. odanacatib), Tibolone, calcitonin or a calcitriol; and hormone replacement therapy. In some embodiments, the second bone-enhancing agent includes, but is not limited to, parathyroid hormone (PTH) or a peptide fragment thereof, PTH-related protein (PTHrp), bone morphogenetic protein, osteogenin, NaF, a PGE2 agonist, a statin, strontium ranelate, and a sclerostin inhibitor (e.g., an anti-sclerostin antibody described in, for example, U.S. Pat. No. 7,592,429 or 7,872,106). In some embodiments, the second bone-enhancing agent is Forteo® (Teriparatide), Preotact®, or Protelos®. In some embodiments, the second bone-enhaiving agent comprises a bone morphogenetic protein (e.g., BMP-1, BMP-2, BMP-3, BMP-4, BMP-5, BMP-6, BMP-7, BMP-8, BMP-9, BMP-10, BMP-11, BMP-12, BMP-13, BMP-14 and/or BMP-15).

In some embodiments, the combination therapy employing an antibody described herein may precede or follow administration of additional therapeutic(s) (e.g., second bone-enhancing agent) by intervals ranging from minutes to weeks to months. For example, separate modalities are administered within about 24 hours of each other, e.g., within about 6-12 hours of each other, or within about 1-2 hours of each other, or within about 10-30 minutes of each other. In some situations, it may be desirable to extend the time period for treatment significantly, where several days (2, 3, 4, 5, 6 or 7 days) to several weeks (1, 2, 3, 4, 5, 6, 7 or 8 weeks) lapse between the respective administrations of different modalities. Repeated treatments with one or both agents/therapies of the combination therapy is specifically contemplated.

### Maintenance Therapeutic Regimen

Also contemplated is the use of a second bone-enhancing agent and/or antibody described herein in a maintenance regimen to, e.g., prevent or slow the loss of bone mineral density. In this regard, a method or use described herein optionally comprises administering one or more amounts of a second bone-enhancing agent effective to maintain bone mineral density for a maintenance period of about 1 week to about 5 years after the treatment period with the antibody has ended. For example, in some embodiments, a method or use described herein comprises the administration of a second bone-enhancing agent to the subject for a maintenance period of about at least about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 25 weeks, about 26 weeks, about 27 weeks, about 28 weeks, about 7 months, about 29 weeks, about 30 weeks, about 31 weeks or longer (e.g., about 8 months, about 9 months, about 10 months, about 11 months, about 1 year, about 15 months, about 18 months, about 2 years, about 3 years, about 4 years, about 5 years or longer (e.g., over the lifetime of the subject). In some embodiments, the maintenance period is about 6-12 weeks. In some embodiments, the maintenance period is about 4-12 weeks, or about 1-3 months. In some embodiments, the maintenance period is about 12-20 weeks, or about 3-5 months. In some embodiments, the maintenance period is about 20-32 weeks, or about 5-8 months. In some embodiments, the maintenance period is about 24-36 weeks, or about 6-9 months. In some embodiments, the maintenance period is about 1 year, about 2 years, about 3 years, about 4 years, about 5 years or longer. “Maintaining” bone mineral density includes maintaining similar levels of bone mineral density parameters experienced in the subject that received the antibody treatment.

### Kits

A pharmaceutical composition comprising one or more antibodies described herein may be placed within containers (e.g., vials or syringes), along with packaging material that provides instructions regarding the use of such pharmaceutical compositions. Generally, such instructions will include a tangible expression describing the antibody concentration, as well as within certain embodiments, relative amounts of excipient ingredients or diluents (e.g., water, saline or PBS) that may be necessary to reconstitute the pharmaceutical composition.

### EXAMPLES

### Example 1-Analysis of Romosozumab PARG C-terminal Variant

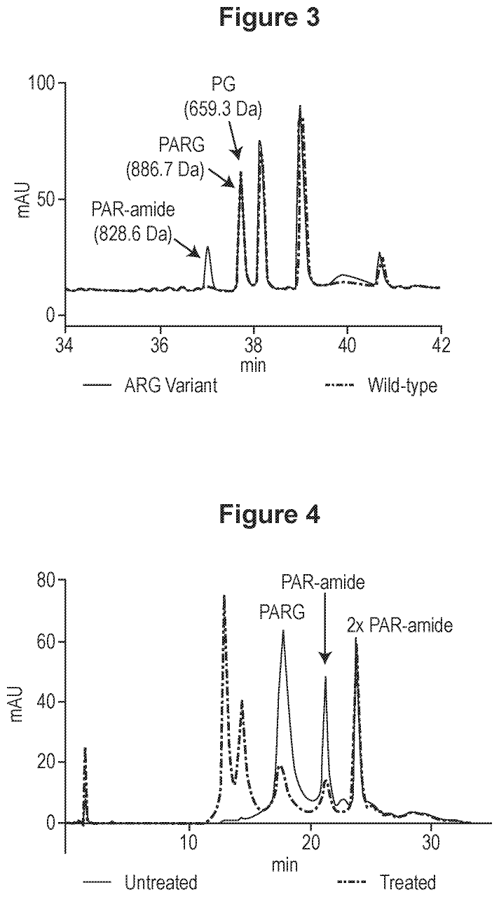

Wild-type romosozumab and a romosozumab PARG C-terminal variant were digested by Lys-C and analyzed by LC/MS peptide mapping. The UV profiles of these two constructs were compared side by side (FIG. 3). It was determined that wild-type romosozumab and the romosozumab PARG C-terminal variant have a similar peak eluting at 37.7 minutes but wild-type romosozumab was determined to have a mass of 659.3 Da and the romosozumab PARG C-terminal variant was determined to have a mass of 886.7 Da. The majority of lysine (K) variants of romosozumab (PGK) was thought to be removed from the process. The presence of a significant amount of the amidated form of the romosozumab PARG C-terminal variant (828.6 Da peak) confirms that the amidation efficiency is sequence dependent when compared to the wild-type romosozumab PG sequence.

Next, the PARG C-terminal variant was then treated with carboxypeptidase (CP-B), analyzed by CEX-HPLC method and compared with the PARG C-terminal variant control which was not treated by the CP-B. There is a significant shift post treatment for the peaks cluting at 17.5 mins and 21 mins, but not for the peak at 24 mins (FIG. 4). It is contemplated that the 24 min peak is the doubly amidated form, which is protected from proteolytic degradation.

### Example 2-C-Terminal Variant Enrichment

Purification or enrichment for different romosozumab species from a composition comprising wild-type romosozumab and the romosozumab PARG C-terminal variant is achieved by Cation Exchange Chromatography (CEX) fractionation. CEX separates proteins based on differences in their surface charges. At a set pH, positively charged variants of wild-type romosozumab are separated on a cation-exchange column (e.g., Dionex Pro Pac WCX-10 analytical column, 2.0 mm×250 mm) and eluted using a salt gradient (e.g., Mobile Phase A: 10:90 (v/v) ACN, 19 mM MES pH 6.2; Mobile Phase B: 10:90 (v/v) ACN, 19 mM MES, 250 mM NaCl, pH 6.2). The different C-terminal variants of romosozumab are charged differently and the more positively charged variant elutes later in CEX. Thus, the elution order is: PG (wild-type), P-amide (amidated proline of wild-type), PARG variant, and PAR-amide. The fraction collector can be programmed to collect CEX eluents containing different variants at different elution times.

### Example 3-Analysis of Romosozumab PARG C-Terminal Variant Aggregation

Without being bound to any particular theory, it is contemplated that because the PARG C-terminal variant is highly charged, such forms would repel non-amidated forms in the compositions, thus reducing aggregation in the composition.

Romosozumab PARG C-terminal variant protein A pool was analyzed side by side with wild-type romosozumab protein A pool using SEC-HPLC, a size exclusion HPLC method that separates protein based on differences in their hydrodynamic volume (Table 1).

TABLE 1

| Molecule | % HMW |
|---|---|
| AMG785 ARG ProA pool | 3.4% |
| AMG785 WT ProA pool | 7.2% |

The data demonstrated that romosozumab PARG C-terminal variant has less high molecular weight species as compared to the wild-type romosozumab.

### Example 4-Viscosity Analysis of Romosozumab PARG C-Terminal Variant

Antibody solutions containing romosozumab PARG C-terminal variant or wild-type romosozumab are measured using a cone and plate. The solutions are concentrated up to 120 mg/mL according to approximate volume depletion, and final concentrations are determined (+10%) using the proteins absorbance at 280 nm (after dilution to end up within 0.1-1 absorbance units (AU)) and a protein specific extinction coefficient. Viscosity analysis is performed on a Brookfield LV-DVIII cone and plate instrument (Brookfield Engineering, Middleboro, MA, USA) using a CP-40 spindle and sample cup or an ARES-G2 rheometer (TA Instruments, New Castle, DE, USA) using a TA Smart Swap 2 degree cone/plate spindle. All measurements are performed at 25° C. and controlled by a water bath attached to the sample cup. Multiple viscosity measurements were collected, manually within a defined torque range (10-90%) by increasing the RPM of the spindle. Measurements are averaged in order to report one viscosity value per sample to simplify the resulting comparison chart.

### Example 5-Solubility Analysis of Romosozumb PARG C-Terminal Variant

To determine the impact of the amino acid variation of romosozumab PARG variant as compared to the wild type romosozumab on solubility upon subcutaneous (SC) injection, a dialysis solubility assay was performed on both wild type and PARG-C terminal variant romosozumab in parallel. This screen entails dialyzing a sample of the romosozumab PARG C-terminal variant and a sample of the wild-type romosozumab into a solution that simulates the pH and ionic strength of the SC space and monitoring the solubility and physical stability of the antibody in these conditions over a short time period. Samples were formulated at ˜63 mg/mL in formulation buffer (pH 5.2). Then each sample was injected into a dialysis cassette and dialyzed into a PBS buffer to mimic the SC space. Visual observations were made 24 hours after initial dialysis. Wild-type romosozumab typically shows precipitation after 24 hours.

The results show that both molecules precipitate in this analysis but the PARG C-terminal variant precipitates less and at a slower rate. This suggests that the variant is more resistant to precipitation than wild type, although the variant does not abolish precipitation completely.

### Example 6-Diffusion Analysis of Romosozumab PARG C-Terminal Variant

To determine the impact of the amino acid variation of romosozumab PARG C-terminal variant as compared to the wild type romosozumab on diffusion from the subcutaneous (SC) space, an assay was performed using Scissor (Pion Inc., Billerica, MA). This assay entails injecting the samples (the romosozumab PARG C-terminal variant or wild-type romosozumab) at ˜70 mg/mL into a simulated SC space comprised of a collagen and hyaluronic acid matrix. The antibody is able to diffuse out of this matrix through a dialysis membrane into a reservoir of carbonate buffer at pH 7.4. Time points were collected for up to 3 days and each time point was assayed for protein concentration by RP-HPLC. The protein concentration vs. time curves generated simulate the diffusion rates from the SC space. In addition, precipitation in the SC matrix is monitored with visual inspection.

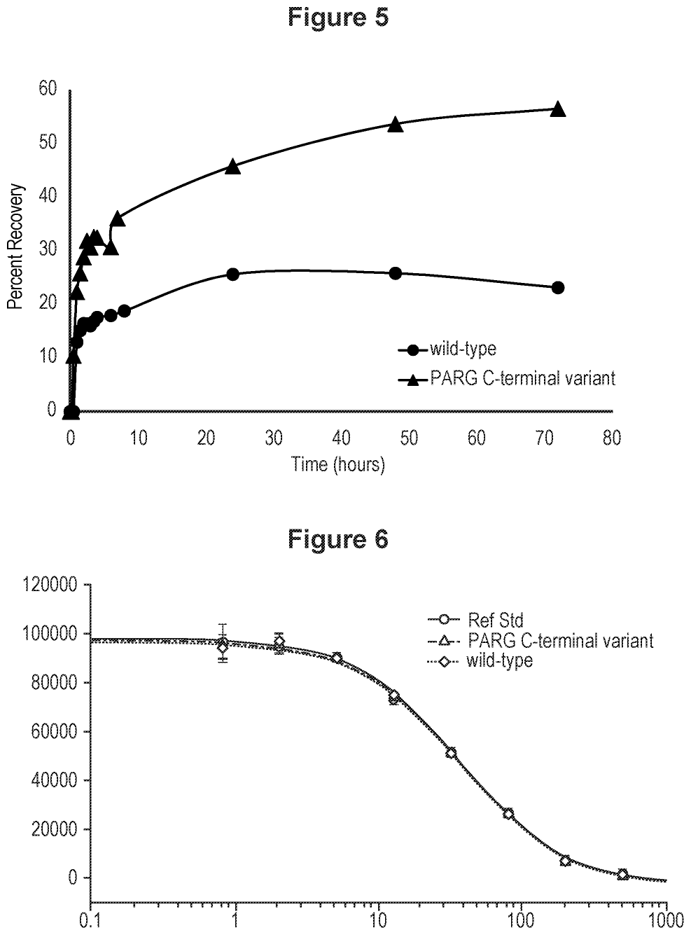

Both the wild type and PARG C-terminal variant romosozumab were tested in the Scissor as described above. The results shown in FIG. 5 indicate that wild type romosozumab diffuses from the simulated SC space at a much lower rate and more wild type romosozumab is retained at the simulated injection site than PARG C-terminal variant romosozumab.

### Example 7-FcRn Binding

FcRn, the neonatal Fc receptor, is an MHC class I-like heterodimer composed of a transmembrane α chain (homologous to MHC class-I like molecules) and a β2 microglobulin light chain. FcRn binds to the interface between C_{H}2 and C_{H}3 domains of IgG heavy chains in the Fc region of the IgG molecule under mildly acidic conditions (˜pH 6) and releases it at neutral pH (˜7.4). By this highly pH-dependent interaction, FcRn mediates IgG homeostasis in human adults by maintaining serum IgG levels.

A competitive binding assay, the AlphaScreen® binding assay (PerkinElmer, San Jose, CA), was used to assess the binding of the Fc domain of wild-type romosozumab and romosozumab PARG C-terminal variant to FcRn. The assay is a bead based amplified luminescent proximity homogeneous assay (“Alpha”) that detects bimolecular interactions. The assay contains two bead types, an acceptor bead and a donor bead. The acceptor beads are coated with a hydrogel that contains thioxene derivatives, as well as nickel chelate which binds to the histidine domain of histidine labeled FcRn (FcRn-His). The donor beads are coated with a hydrogel that contains phthalocyanine, a photosensitizer, and streptavidin, which binds to biotinylated CHO derived human Fc. When FcRn-His and the biotinylated human Fc bind together, they bring the acceptor and donor beads into close proximity. When laser light is applied to this complex, ambient oxygen is converted to singlet oxygen by the donor bead. If the beads are in close proximity, an energy transfer to the acceptor bead occurs, resulting in light production (luminescence), which is measured in a plate reader equipped for AlphaScreen® signal detection.

When an antibody is present at sufficient concentrations to inhibit the binding of FcRn-His to the biotinylated human Fc domain, a dose dependent decrease in emission at 570 nm is observed. The test sample binding relative to the antibody reference standard is determined and reported as % relative binding and can be used to demonstrate the integrity of the Fc domain of the antibody. It is contemplated that compositions having the PARG C-terminal variant will have a similar or better dose response curve than the wild type antibody.

The results are shown in FIG. 6. It was observed that both wild type romosozumab and PARG C-terminal variant romosozumab bound FcRn similarly and FcRn binding was not affected by the PARG mutation.

### Example 8-FcγRIIa Binding

FcγRIIa is an activating Fc receptor expressed on monocytes, certain dendritic cells, neutrophils, B cells, platelets and NK cells. FcγRIIa (CD32a) is the most widely distributed FcγR with two extracellular Ig-like domains and low binding affinity for monomeric IgG. There are two common allelic variants in humans that are known to exist for FcγRIIa, expressing either histidine or arginine at position 131 (131H and 131R, respectively).

A competitive binding assay was developed to assess the binding of wild-type romosozumab and romosozumab PARG C-terminal variant to FcγRIIa (131H). The FcγRIIa (131H) binding assay is a bead-based amplified luminescent proximity homogencous assay (AlphaScreen® binding assay (PerkinElmer, San Jose, CA) that detects bimolecular interactions. The assay contains 2 bead types, an acceptor bead and a donor bead. The acceptor beads contain the fluorophore europium chelate and are coated with a hydrogel that contains glutathione, which binds recombinant human FcγRIIa (131H)-glutathione-S-transferase (FcγRIIa (131H)-GST). The donor beads are coated with a hydrogel that contains phthalocyanine, a photosensitizer, and streptavidin, which binds to biotinylated human IgG1. When FcγRIIa (131H)-GST and the biotinylated human IgG1 bind together, they bring the acceptor and donor beads into proximity. When a laser is applied to this complex, ambient oxygen is converted to singlet oxygen by the donor bead. When the acceptor and donor beads are near, the singlet oxygen diffuses within the acceptor beads resulting in light production (luminescence), which is measured in a plate reader equipped for luminescence signal detection.

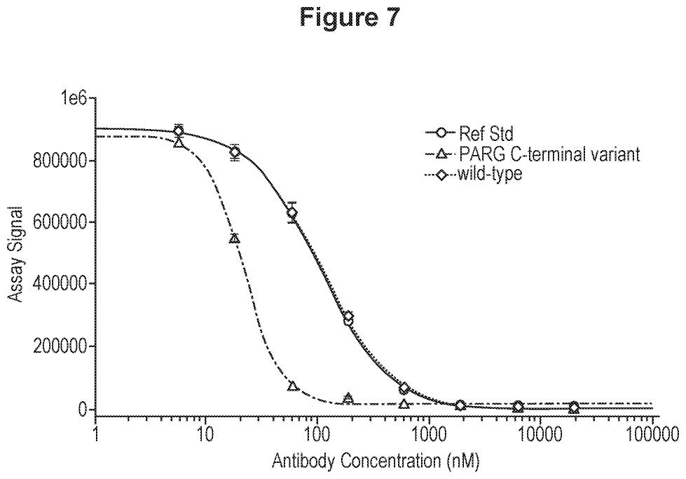

When antibody is present at sufficient concentrations to inhibit the binding of FcγRIIa (131H)-GST to the biotinylated human IgG1, a dose-dependent decrease in emission at 570 nm is measured. The test sample binding relative to the antibody reference standard is determined and reported as % relative binding and can be used to demonstrate the integrity of the Fc domain of the antibody. The results are shown in FIG. 7. It was observed that the relative binding of PARG C-terminal variant romosozumab to FcγRIIa (131H) was much higher than wild-type romosozumab.

### Example 9-Mouse Pharmacokinetic Study

To evaluate in vivo drug exposure and bioavailability, a single dose pharmacokinetic study in mice is performed. Romosozumab PARG C-terminal variant is injected either intravenously (via tail vein) or subcutaneously at a dose of 1 mg/kg. Using nine animals per group, staggered sampling permits collection of data at a large number of time points without exceeding the maximum volume of blood that can be drawn from an individual animal. At each time point, 0.05 ml of blood is drawn. Animals 1 to 3 are sampled at 0.083, 24, 96 and 192 hours post-dose. Animals 4-6 are sampled at 1, 48, 168 and 240 hours. Animals 7-9 are sampled at 6, 72 and 192 hours. Serum is collected from the whole blood sample and test article concentration is determined by a binding immunoassay such as an ELISA (Enzyme-Linked ImmunoSorbant Assay). Changes in test article concentration over time can be used to calculate pharmacokinetic parameters via two compartment analysis. Parameters of interest include, but not limited to, area under the plasma concentration-time curve (AUC), half-life (t_{1/2}) and clearance (CL) for each dose group. Bioavailability can be determined as the ratio of AUC for the subcutaneous dose to the AUC for the intravenous dose.

1. An antibody that specifically binds to sclerostin of SEQ ID NO: 1 and comprises a set of six CDRs set forth in SEQ ID NOs: 2-7, wherein the antibody comprises a heavy chain comprising the amino acid sequence Pro-Ala-Arg-Gly (SEQ ID NO: 8) at the C-terminus of the heavy chain.

2. (canceled)

3. The antibody of claim 1, wherein the C-terminus of the heavy chain is amidated.

4. The antibody of claim 1, wherein the C-terminus of both heavy chains comprises the amino acid sequence Pro-Ala-Arg-Gly (SEQ ID NO: 8).

5. The antibody of claim 1, wherein the C-terminus of both heavy chains is amidated.

6. The antibody of claim 1, comprising the light chain amino acid sequence set forth in SEQ ID NO: 12 and heavy chain amino acid sequence set forth in SEQ ID NO: 13 or SEQ ID NO: 14.

7. The antibody of claim 1, wherein the antibody comprises a sequence of amino acids comprising Pro-Ala-Arg-Gly-Lys (SEQ ID NO: 11) at the C-terminus of a heavy chain.

8. The antibody of claim 7, comprising the light chain amino acid sequence set forth in SEQ ID NO: 12 and the heavy chain amino acid sequence set forth in SEQ ID NO: 14.

9. A pharmaceutical composition comprising a population of antibodies of claim 1 and a pharmaceutically acceptable carrier.

10. A pharmaceutical composition comprising a mixture of antibodies that specifically bind to sclerostin of SEQ ID NO: 1 and a pharmaceutically acceptable carrier;
wherein about 3-5% of the antibodies in the composition are a population of antibodies of claim 1.

11. The pharmaceutical composition of claim 10, wherein all or part of the population of antibodies comprise a single heavy chain comprising a C-terminal Pro-Ala-Arg-Gly (SEQ ID NO: 8) sequence.

12. The pharmaceutical composition of claim 10, wherein all or part of the population of antibodies comprise a heavy chain comprising a C-terminal Pro-Ala-Arg-Gly (SEQ ID NO: 8) sequence that is amidated.

13. The pharmaceutical composition of claim 12, wherein less than about 35% of the population of antibodies is singly amidated.

14. The pharmaceutical composition of claim 10, wherein all or part of the population of antibodies comprise a C-terminal Pro-Ala-Arg-Gly (SEQ ID NO: 8) sequence in both heavy chains.

15. The pharmaceutical composition of claim 14, wherein all or part of the population of antibodies comprising two heavy chains comprising a C-terminal Pro-Ala-Arg-Gly (SEQ ID NO: 8) sequence are amidated on both heavy chains.

16. The pharmaceutical composition of claim 15, wherein less than about 35% of the population of antibodies are amidated on both heavy chains.

17. The pharmaceutical composition of claim 9, wherein less than about 35% of the population of antibodies comprise heavy chains that are not amidated.

18. The pharmaceutical composition of claim 9, wherein about 33% of the population of antibodies are not amidated, about 33% of the population of antibodies comprise one amidated heavy chain, and about 33% of the population of antibodies comprise two amidated heavy chains.

19. The pharmaceutical composition of claim 9, further comprising a calcium salt, an acetate buffer, a polyol and a surfactant.

20. (canceled)

21. (canceled)

22. (canceled)

23. (canceled)

24. The pharmaceutical composition of claim 9, further comprising 55 mM acetate, 13 mm calcium, 6.0% (w/v) sucrose, 0.006% (w/v) polysorbate 20, pH 5.2.

25. A method of increasing bone mineral density in a subject in need thereof comprising administering the composition of claim 9 to the subject in an amount effective to increase bone mineral density in the subject.


SEQUENCE LISTING (16 sequences)

SEQ ID NO: 1 - protein, 190 aa, Homo sapiens
  REGION 1..190: note = MISC_FEATURE - Human Sclerostin
QGWQAFKNDATEIIPELGEYPEPPPELENNKTMNRAENGGRPPHHPFETKDVSEYSCRELHFTRYVTDGPCRSAKPVTELVCSGQCGPARLLPNAIGRGKWWRPSGPDFRCIPDRYRAQRVQLLCPGGEAPRARKVRLVASCKCKRLTRFHNQSELKDFGTEAARPQKGRKPRPRARSAKANQAELENAY

SEQ ID NO: 2 - protein, 5 aa, Mus musculus
  REGION 1..5: note = MISC_FEATURE - romo HCDR1
DYNMH

SEQ ID NO: 3 - protein, 17 aa, Mus musculus
  REGION 1..17: note = MISC_FEATURE - romo HCDR2
EINPNSGGAGYNQKFKG

SEQ ID NO: 4 - protein, 14 aa, Mus musculus
  REGION 1..14: note = MISC_FEATURE - romo HCDR3
LGYDDIYDDWYFDV

SEQ ID NO: 5 - protein, 11 aa, Mus musculus
  REGION 1..11: note = MISC_FEATURE - romoLCDR1
RASQDISNYLN

SEQ ID NO: 6 - protein, 7 aa, Mus musculus
  REGION 1..7: note = MISC_FEATURE - romo LCDR2
YTSRLLS

SEQ ID NO: 7 - protein, 9 aa, Mus musculus
  REGION 1..9: note = MISC_FEATURE - romo LCD3
QQGDTLPYT

SEQ ID NO: 8 - protein, 4 aa, Homo sapiens
  REGION 1..4: note = MISC_FEATURE - C-Terminal variant sequence
PARG

SEQ ID NO: 9 - protein, 107 aa, Mus musculus [sequence omitted]

SEQ ID NO: 10 - protein, 123 aa, Mus musculus [sequence omitted]

SEQ ID NO: 11 - protein, 5 aa, Homo sapiens
  REGION 1..5: note = MISC_FEATURE - C-terminal variant sequence
PARGK

SEQ ID NO: 12 - protein, 236 aa, synthetic construct
  REGION 1..236: note = Humanized antibody sequence
  REGION 1..236: note = MISC_FEATURE - romo light chain
MDMRVPAQLLGLLLLWLRGARCDIQMTQSPSSLSASVGDRVTITCRASQDISNYLNWYQQKPGKAPKLLIYYTSRLLSGVPSRFSGSGSGTDFTLTISSLQPEDFATYYCQQGDTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 469 aa, synthetic construct
  REGION 1..469: note = Humanized antibody sequence
  REGION 1..469: note = MISC_FEATURE - romo heavy chain variant without lysine
MDWTWRILFLVAAATGAHSEVQLVQSGAEVKKPGASVKVSCKASGYTFTDYNMHWVRQAPGQGLEWMGEINPNSGGAGYNQKFKGRVTMTTDTSTSTAYMELRSLRSDDTAVYYCARLGYDDIYDDWYFDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPARG

SEQ ID NO: 14 - protein, 470 aa, synthetic construct
  REGION 1..470: note = Humanizedantibody sequence
  REGION 1..470: note = MISC_FEATURE - romo heavy chain variant with lysine
MDWTWRILFLVAAATGAHSEVQLVQSGAEVKKPGASVKVSCKASGYTFTDYNMHWVRQAPGQGLEWMGEINPNSGGAGYNQKFKGRVTMTTDTSTSTAYMELRSLRSDDTAVYYCARLGYDDIYDDWYFDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPARGK

SEQ ID NO: 15 - protein, 26 aa, Homo sapiens
  REGION 1..26: note = MISC_FEATURE - amino acids 86-111 of SEQ ID NO: 1
CGPARLLPNAIGRGKWWRPSGPDFRC

SEQ ID NO: 16 - protein, 468 aa, synthetic construct
  REGION 1..468: note = Humanized Antibody sequence
  REGION 1..468: note = MISC_FEATURE - romo heavy chain wild type
MDWTWRILFLVAAATGAHSEVQLVQSGAEVKKPGASVKVSCKASGYTFTDYNMHWVRQAPGQGLEWMGEINPNSGGAGYNQKFKGRVTMTTDTSTSTAYMELRSLRSDDTAVYYCARLGYDDIYDDWYFDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK